(19) United States
(12) Patent Application Publication
(10) Pub. No.: US 2019/0114715 A1
(43) Pub. Date: Apr. 18, 2019

(54) METHOD AND SYSTEM FOR PROCESSING PET INSURANCE CLAIMS

(71) Applicant: Cleverland Holding LLC, Cleveland, OH (US)

(72) Inventors: Richard Hessinger, Berea, OH (US); Nicolaas Lindsey, Medina, OH (US); Aaron Puma, Parma, OH (US); Melissa Ing, Stow, OH (US); Ambrish Jaiswal, Beachwood, OH (US)

(21) Appl. No.: 16/161,886

(22) Filed: Oct. 16, 2018

Related U.S. Application Data
(60) Provisional application No. 62/573,335, filed on Oct. 17, 2017.

(51) Int. Cl. G06Q 40/08; G16H 10/60; G06N 20/00

(52) U.S. Cl. CPC G06Q 40/08; G06N 20/00; G16H 10/60

(57) ABSTRACT

A system and method for auto-adjudication of pet insurance claims and precertifications includes receiving user sign on information, sending the user sign on credentials over a communication network to a central cloud server where they are used to retrieve the match hospital's database pool. The hospital database pool is searched for an invoice matching the claim information sent or an estimate matching the precertification information sent with the user credentials and is retrieved and transmitted from the central cloud server to a machine-learning model in a backend system server. The machine-learning model electronically processes the invoice or estimate information by marking each item on the invoice or estimate with a coverage type, wherein the marking is based upon pre-determined input conditions and machine learned data of prior invoices and estimates, the total is calculated in the business rule engine, wherein the total covered amount is the agreed upon amounts for each approved item, the agreed upon amount stored in the backend system server databases. A payment is initiated to the hospital based upon the calculated total covered amount and notice of payment to hospital is sent and displayed on the user interface for claims. A notice of approval is initiated to the hospital based upon the calculated total approved amount and displayed on the user interface for precertifications.

### CROSS-REFERENCE TO RELATED APPLICATION

This application claims the benefit of U.S. Provisional Patent Application No. 62/573,335, filed on Oct. 17, 2017, the disclosure of which is incorporated by reference herein in its entirety.

### FIELD OF INVENTION

The present disclosure is directed to a system and method for providing pet insurance. More particularly, the present disclosure is directed to a method and communication architecture used within a pet insurance system allowing users to access a patient's records, and to process claims in real-time.

### BACKGROUND

Systems and methods for registering new patients for pet insurance and processing claims against a particular insurance policy are known. However, these systems do not allow a pet parent, veterinary clinic, or hospital the ability to process claims in real-time. Current systems use, for example, APIs for claim submissions. However, these systems require large teams of people to receive, review, and code these claims for processing. Thus, while a team of people may review the claim soon after submission, it is not reviewed and processed in real-time. A system employing traditional algorithmic solutions requires significant computing resources, and thus operates slowly and/or consumes a high level of electric power to process.

Additionally, current systems utilize a two-tier system where a first tier is the veterinary practice and a second tier is the insurance company's internal system. Current systems allow access to claim records, enrollment records, and claims processing to be handled by a single tier while the second tier contain the veterinary records.

### SUMMARY OF THE INVENTION

The present system and method uses machine-learning algorithms to auto-adjudicate claims in real time. Such machine-learning techniques reduces the processing time of the computer resources being employed and thus draws significantly less electric power.

Additionally, the present system and method utilizes three tiers to store and process the information. The use of a third tier for storing and processing the information results in improved efficiency of the existing two-tiered systems. In other words, the computer systems of veterinary practices and insurance companies perform fewer processing functions related to insurance claims.

In one embodiment, a computer-implemented method for auto-adjudication of a pet insurance claims and precertifications is disclosed. The method includes displaying on a user computer screen a user web interface configured to receive user sign on credentials for a pet insurance claims system. The user sign on credentials include a user name, a hospital location, customer information, and request information related to one of the group consisting of a pet insurance claim and a pet insurance precertification. The method further includes sending the user sign on credentials including the user name, the hospital location, the customer information, and the request information over a communication network from a veterinary hospital. The user sign on credentials are sent to a central cloud server to retrieve a matching hospital database pool. The method further includes retrieving from the matching hospital database pool one of an invoice matching the pet insurance claim and an estimate matching the pet insurance precertification, wherein the one of the invoice and the estimate includes a plurality of item types. The method also includes transmitting the request information from the central cloud server to a machine-learning API in a backend system server. The method further includes electronically processing the one of the invoice and the estimate in the machine-learning API by marking each item type in the one of the invoice and the estimate. The marking is based upon pre-determined input conditions and data from prior claims and precertifications to create a machine-learning model. The method also includes electronically processing the one of the pet insurance claim and the pet insurance precertification in a business rules engine and calculating a total amount. The total amount is an agreed upon amounts for each item approved by the business rules engine during the step of electronically processing. The method further includes storing the agreed upon amount in a backend system server database, initiating a payment to the hospital location based upon the calculated total amount, and displaying on the user computer screen, the calculated total amount and notice of payment to the hospital location for approved claims.

In another embodiment, a system for auto-adjudication of pet insurance claims or precertification includes a communication network configured to send and receive information. The system also includes a central cloud server system including a hospital database pool configured to store policyholder information pertaining to customers and pets, a system API, and a web portal interface used to interact with databases in the central cloud server system. The system also includes a backend server system comprising a machine-learning API and a machine-learning model configured to store one or more pieces of information concerning veterinary treatments or procedures classified as approved, a business rule engine used to validate and process claim or precertification information upon receipt in real time based upon the approved veterinary treatments or procedures. The system further includes a pet hospital server system comprising one or more computers with a display connected to a Practice Information Management System (PIMS) database configured to store pet hospital customer records and pet medical and billing records.

In yet another embodiment, a computer readable medium with computer executable instructions performs the step of receiving information including a user name, a hospital location, customer information, and request information related to one of the group consisting of a pet insurance claim and a pet insurance precertification. The computer readable medium also performs the step of sending the user sign on credentials including the user name, the hospital location, the customer information, and the request information over a communication network from a veterinary hospital. The user sign on credentials are sent to a central cloud server to retrieve a matching hospital database pool. The computer readable medium further performs the step of retrieving from the matching hospital database pool one of an invoice matching the pet insurance claim and an estimate matching the pet insurance precertification. The one of the invoice and the estimate includes a plurality of item types. The computer readable medium also performs the step of transmitting the request information from the central cloud server to a machine-learning API in a backend system server. The computer readable medium further performs the step of electronically processing the one of the invoice and the estimate in the machine-learning API by marking each item type in the one of the invoice and the estimate. The marking is based upon pre-determined input conditions and data from prior claims and precertifications to create a machine-learning model. The computer readable medium also performs the step of electronically processing the one of the pet insurance claim and the pet insurance precertification in a business rules engine. The computer readable medium further performs the step of calculating a total amount, wherein the total amount is an agreed upon amounts for each item approved by the business rules engine during the step of electronically processing. The computer readable medium also performs the step of storing the agreed upon amount in a backend system server database, and initiating a payment to the hospital location based upon the calculated total amount.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates a high-level view of the exemplary system layout;

FIG. 2 illustrates a detailed block diagram of each tier of the exemplary system;

FIG. 3 illustrates a representative administrator user interface;

FIG. 4 is a flow chart illustrating an exemplary method or processing claims using machine-learning mechanisms.

### DETAILED DESCRIPTION

The disclosure is described in the context of utilizing cloud computing but it should be understood that other remote systems may be implemented. Each database should be understood to include memory configured to store data, sent and received by each database, for the system.

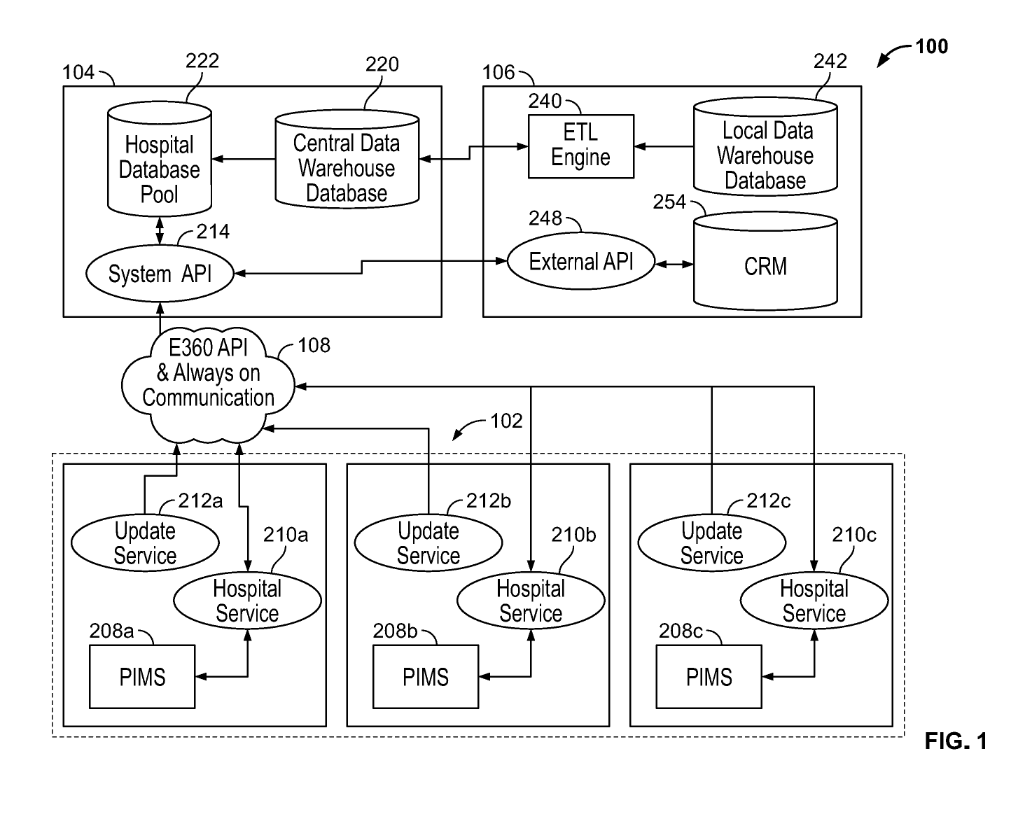

FIG. 1 illustrates a high-level view of the exemplary system. The system 100 comprises three main tier elements, a hospital server block 102, a central cloud server block 104, and backend system server block 106, connected over a communication network 108. A hospital server block 102 is the first tier element, and is maintained at a customer location. The system 100 supports at least one hospital server block 102, wherein each hospital server block 102 contains a Practice Information Management System (PIMS) 208, and multiple services 210, 212 with processors and memory to communicate with the rest of the system. Each system 100 can support up to N number of hospital server blocks 102.

A central cloud server block 104 is the second tier element, and is maintained by an installation company and located in the cloud. The central cloud server block 104 contains multiple databases and portals used in storing, sending, and receiving claim and hospital information 220, 222, 214.

A backend system server block 106 is the third tier element, and is maintained by the installation company. All communications coming from a System Application Programming Interface (API) 214 to the system server block 106 come through an external API 248. The external API 248 integrates with internal applications and saves to a Customer Relationship Management (CRM) database 254. The backend system server 106 maintains copies of the hospital information, enrolls new clients, and, using machine-learning mechanisms, processes claims and precertifications in real time 240, 242, 248, 254.

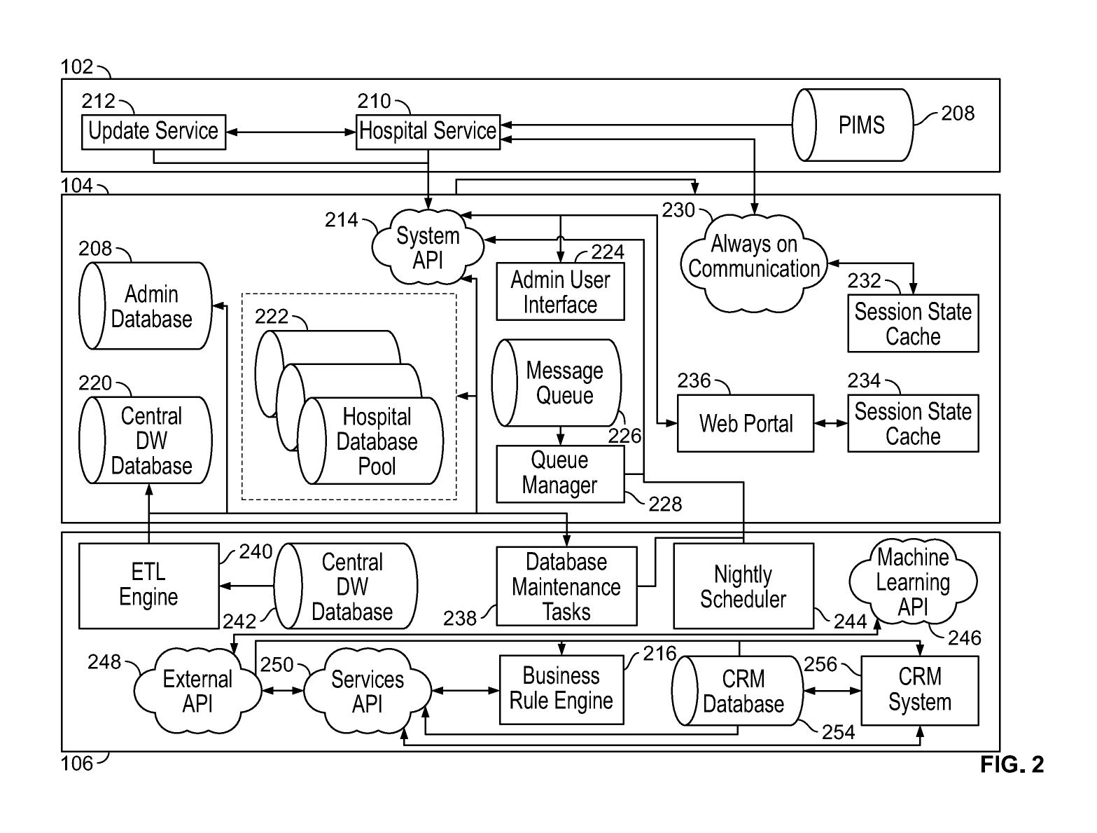

FIG. 2 illustrates a detailed block diagram of each tier of the exemplary system 100. In the illustrated block diagram, the system 100 is divided into three major server blocks 102, 104, 106, where each block is located remotely from the remaining server blocks. The hospital server block 102 is located in offices and hospitals. The goal of the hospital server block 102 is to take Practice Information Management System (PIMS) data from clients and post it to a central cloud system. There can be more than one hospital server block 102 within a single system, each functioning independently from the other hospital server blocks.

Each hospital server block 102 includes a PIMS 208, which stores the hospital's medical and billing records for pets. The hospital service 210 is connected to the PIMS database 208 and its primary purpose is to extract data from the PIMS database 208 and transmits the data over a communication network 108 to a system API 214 in the central cloud server block 104 using a communication service, such as the JAGUAR DATA SERVICE available from VETDATA. The hospital service 210 executes iterative data pulls happening at a time specified in the settings data file at the hospital. Nightly data pulls are done through the nightly scheduler 244. The PIMS database 210 regularly synchronizes with the system API 214 to maintain up-to-date databases and invoice information. The update service 212 connects to the hospital service 210 to verify that the version of service remains up to date and checks that the service is running properly. The update service 212 will also restart the hospital service 210.

The central cloud server block 104 contains databases and services maintained remotely in the cloud. These databases are used to disseminate the PIMS data and link it with client data maintained in the central server in the hospital database pools 222 using a system API 214. The database pools 222 are a grouping of databases that are billed together with a shared allowance of billing units. The benefit to having the pools, aside from billings, is that databases in a pool can access other databases that are referenced. The exemplary system described herein can have multiple pools and each pool can contain a shared resource pool. Data is kept on the hospital database pools 222 where internally developed APIs can access it. The central cloud server block 104 is comprised of servers and databases that maintain copies of the server records and hospital records, and communicates with both the hospital servers 102 and the backend system servers 106. This information is mapped in the hospital database, as part of the database pools 222. The business rule engine 216, contained inside the backend system server 106, is used to run policy-rating rules for quotes, claim and precertification validation rules for submitted claims. The data generated from the business rule engine 216 is sent back through the services API 250 and the external API 248 to the CRM database 254.

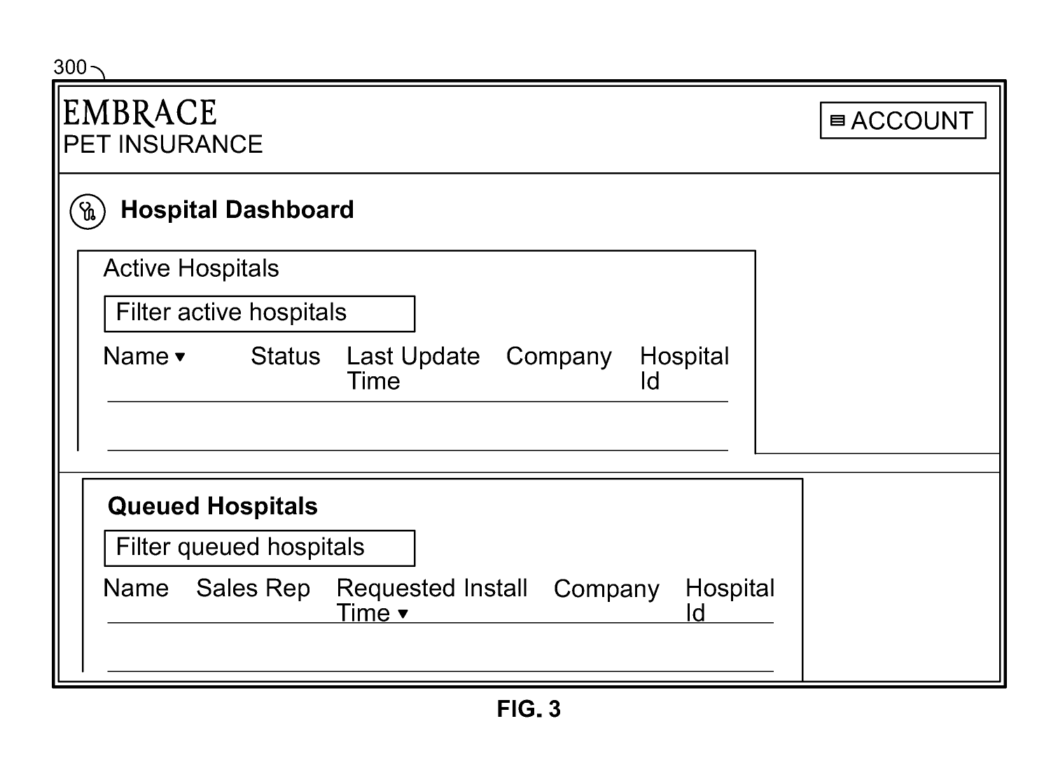

Turning to, FIG. 3, FIG. 3 illustrates a representative admin user interface in the form of a Graphical User Interface (GUI) 300 that is populated with information from the admin User Interface (UI) database 224. Users complete all user and hospital management through the admin UI database 224 and GUI. User accounts can be added, updated, deleted, and hospital accounts can be linked to the hospital service 210. All hospital service configurations are managed through the Admin UI 224.

Returning to FIG. 2, the Queue Manager 228 manages the nightly and iterative data processing. The initial iterative data extractions and the message IDs are dropped into the Message Queue Manager 226. The Queue Manager 228 disseminates the data to the individual hospital records in the hospital database pool 222. The web portal 236 is used to interact with the databases in the central cloud server block and the web portal has credentials to call the system API 214. The Always On Communication 230 allows commands sent from the admin UI 224 to the hospital service and receive a response.

Data retrieval is initiated using settings created in the Admin UI 224 and stored in the Admin database 218. The always on communications 230 and communication session state cache 232 work together to connect to the hospital service 210 on the hospital client computers. Using a communication protocol, such as JAGUAR SDK, data is retrieved from the PIMS 208 and uploaded to the admin database 218 and inserted in the message queue 226 via the system API 214. The queue manager 228 assists in uploading the data into the proper database in the hospital database pool 222 through the database maintenance tasks 238.

The central data warehouse database 220 maintains specific details regarding each hospital account such as the primary contact information, sales rep information, contract dates, etc. A subset of the information maintained in the data warehouse database 242 located in the backend system server block 106 is also uploaded to the central data warehouse database 220.

The backend system server block 106 is maintained onsite by the insurance carrier's offices. It includes databases and schedules to check and update the hospital and patient information and contains the information necessary to determine claim and precertification processing. It should be understood that the databases maintained in this server block contain processors and the necessary memory with computer code configured to assist in the processing of data.

The Extract, Transform, Lead (ETL) engine 240 uses three ETL packages to transfer data from the local data warehouse database 242 in the backend system server block 106 to the Central data warehouse database 220 located in the central cloud server block 104. The local data warehouse database 242 maintains a copy of all the policyholder information such as the pet name, contact information, type of pet(s), policy version, etc. This information is also duplicated in the CRM database 254 and for performance reasons. A nightly scheduler 244 initiates a nightly task that transfers data from the CRM database 254 to the local data warehouse database 242. The ETL engine 240 handles the moving of data from the CRM database 254 to the local data warehouse 242. The local data warehouse database 242 is utilized as a reporting database to ensure that data is not corrupted or lost in the CRM database 254. The CRM database 254 is the main database utilized by the CRM system 256. The CRM system 256 is the policy and claim management system.

The database maintenance tasks 238 works with the nightly scheduler 244 to update the data in the hospital database pools 222. Every night, the nightly scheduler 244 initiates a nightly sync from the hospital PIMS 208 to the central cloud server block 104 while another nightly task sends data from the local data warehouse database 242 to the central cloud server block 104.

The exemplary system can be used to establish new clients within the pet insurance system. To initiate a new client, a hospital staff member logs into the web portal 236 using a unique organization code and pin number. The web portal 236 sends a unique hospital ID that is used by the system API 214 to determine if the user is an authorized user. The system API 214 pulls the hospital specific information from the hospital database pool 222. Web portal session state cache 234 is used to scale out the web portal 236 for higher loads. It maintains the logged in users session as it transitions between scaled out instances.

The web portal 236 communicates with the system API 214 to access the records for the specific hospital. Through the web portal 236, the user can see the hospital's daily appointment schedule, create quotes for non-insured pets, and view policy details and submit claims and precertifications for insured pets. A user can select a particular pet in the hospital's database pool 222 and auto-populates a form for a user through the web portal 236. The user then fills out additional information necessary for a quote, such as name, address, pet details, age, breed, etc. After the user fills out this information, the form is sent to the backend system server block 106, and the services API 250 generates the quote. The quote is stored in the CRM database 254 within the backend system server block 106, and sent to the pet owner directly. With the described method and system, the hospital cannot directly issue pet insurance to a pet owner. Each emailed quote contains a link to allow the pet parent to view the quote. If a pet parent chooses to purchase insurance (not shown), the pet parent would have to either contact a service center to speak with a service representative or access the system website to complete the quote and purchase.

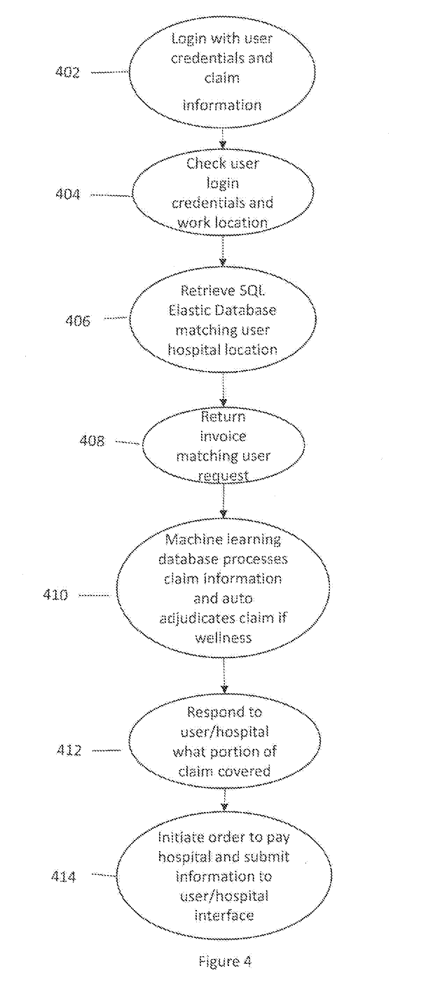

With respect to FIG. 4, an exemplary method for processing a claim or precertification is shown. An insurance claim represents a claim for payment for a service that has been rendered. A precertification represents an approval to cover payment for a future service. The same system may be used to process both claims and precertifications. In instances where a precertification has been auto-adjudicated before the service has been rendered, a claim may be submitted after the service has been performed.

The system and method handles multiple types of insurance claims and precertifications. Auto-adjudication of claims and precertifications can occur for wellness claims or illness claims. A “wellness claim” is directed to routine care for an animal, such as a checkup. Other claims for accidents or illnesses are non-wellness claims. An “illness claim” is directed to care for an animal for acute or chronic illness such as infections, respiratory disorders, etc.

To initiate claim or precertification processing, a user at the hospital logs into the system through the web portal 236 and submits a claim or precertification 402. The user credentials are sent to the system API and checked to confirm where the user works 404. A Structured Query Language (SQL) elastic database that matches the hospital where the user works is retrieved 406. The SQL database is searched to find the invoice or estimate that matches the claim or precertification submitted by the user and the matching invoice or estimate is returned to the user 408. The claim or precertification is processed by the machine-learning API 246 to determine if it includes wellness or illness items 410. The machine-learning API 246 is populated with basic wellness and illness items and claim information; however, the model is continually updated to accommodate newly classified wellness or illness items. When the machine-learning API 246 receives a claim or precertification, it marks each item type in the invoice or estimate information wherein the marking is based upon pre-determined input conditions and data from prior claims and precertifications to create a machine-learning model.

If the machine-learning API 246 determines that the claim includes all wellness line items or if the pet was underwritten to be a healthy animal, the invoice and claim or estimate and precertification pass through the business rules engine and are auto-adjudicated. Auto-adjudication allows the hospital to be immediately informed of how much is covered by insurance on each invoice or how much will be covered for an estimated procedure through the web interface 412. An order to pay the hospital 414 is generated for claims and the hospital is paid directly within a few days. For all auto adjudicated claims, payment is immediately scheduled thus alleviating the need for the hospital or customer to submit claim information later. For any claim that is not auto-adjudicated, the customer must pay out of pocket but can still submit claim information to determine if reimbursement is allowed.

If the claim or precertification is not a wellness claim, the user is prompted to enter additional information into the web portal 236, such as diagnoses information. The hospital user can input information regarding the items classified as non-wellness or non-illness but these claims are not auto-adjudicated and are processed later. The customers pay non-auto adjudicated claims as usual and, if a claim is approved, the customer receives payment later. Precertifications are approvals of an estimated procedure to be done in the future so no payment is exchanged.

The above merely illustrates the principles of the invention. It is thus appreciated that those skilled in the art will be able to devise various arrangements, which, although not explicitly described or shown herein, embody the principles of the invention and are included within its spirit and scope. Furthermore, all examples and conditional language recited herein are principally intended expressly to be only for pedagogical purposes to aid the reader in understanding the principles of the invention and the concepts contributed by the inventor(s) to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, those skilled in the art will appreciate the block diagrams herein represent conceptual views embodying the principles of the invention. Similarly, it will be appreciated that any flow charts, flow diagrams, and the like represent various processes, which may be substantially represented in computer readable medium and so executed by a computer or processor, whether or not such computer or processor is explicitly shown.

The functions of the various elements shown in the figures, including functional blocks labeled as “servers” or “databases” may be provided by dedicated hardware as well as hardware capable of executing software in association with appropriate software. When provided by a server or computer, the functions may be provided by a single dedicated processor, by a single shared processor, or by a plurality of individual processors, some of which may be shared. Moreover, explicit use of the term “server” or “computer” should not be construed to refer exclusively to hardware capable of executing software, and may implicitly include, without limitation, digital signal processor (DSP) hardware, read-only memory (ROM) for storing software, random access memory (RAM), and non-volatile storage. Other hardware, conventional and/or custom, may also be included.

What is claimed is:

1. A computer-implemented method for auto-adjudication of a pet insurance claims and precertifications, the method comprising:
displaying on a user computer screen a user web interface configured to receive user sign on credentials for a pet insurance claims system, wherein the user sign on credentials include a user name, a hospital location, customer information, and request information related to one of the group consisting of a pet insurance claim and a pet insurance precertification;
sending the user sign on credentials including the user name, the hospital location, the customer information, and the request information over a communication network from a veterinary hospital, wherein the user sign on credentials are sent to a central cloud server to retrieve a matching hospital database pool;
retrieving from the matching hospital database pool one of an invoice matching the pet insurance claim and an estimate matching the pet insurance precertification, wherein the one of the invoice and the estimate includes a plurality of item types;
transmitting the request information from the central cloud server to a machine-learning API in a backend system server;
electronically processing the one of the invoice and the estimate in the machine-learning API by marking each item type in the one of the invoice and the estimate, wherein the marking is based upon pre-determined input conditions and data from prior claims and precertifications to create a machine-learning model;
electronically processing the one of the pet insurance claim and the pet insurance precertification in a business rules engine;
calculating a total amount, wherein the total amount is an agreed upon amounts for each item approved by the business rules engine during the step of electronically processing;
storing the agreed upon amount in a backend system server database;
initiating a payment to the hospital location based upon the calculated total amount; and
displaying on the user computer screen, the calculated total amount and notice of payment to the hospital location for approved claims.

2. The computer-implemented method of claim 1, further including:
transmitting data from the backend system server to a database in the central cloud server to update policyholder information including pet name, contact information, type of pet, and policy version; and
updating information in the backend system server based on a scheduled update, wherein the scheduled update initiates a transfer of data from a main information database to a duplicate database.

3. The computer-implemented method of claim 1, further including retrieving from the matching hospital database pool at least one hospital database matching the hospital location and Practice Information Management System (PIMS) received from the hospital location.

4. The computer-implemented method of claim 1, wherein the one of the pet insurance claim and the pet insurance precertification includes:
customer information, including name;
pet information, including type of pet, age, breed, visit diagnosis;
at least one of a date of service and a future date of service;
at least one of a service provided on the date of service and a service estimate provided for the future date of service; and
cost information for the at least one of the service provided and the service estimate provided.

5. The computer-implemented method of claim 1, wherein the machine-learning model automatically processes claim or precertification information in real-time, upon receipt of the one of the pet insurance claim and the pet insurance precertification, based upon previously provided information.

6. The computer-implemented method of claim 1, further including:
displaying on the user computer screen a request for additional diagnosis information for each claim or precertification identified as illness.

7. The computer-implemented method of claim 1, further including submitting customer information with the user sign on credentials, wherein the customer information is sent to the backend system server to determine if the customer is an enrolled member.

8. The computer-implemented method of claim 1, wherein the backend system server is only accessible through a hospital web interface.

9. A system for auto-adjudication of pet insurance claims or precertification, the system comprising:
a communication network configured to send and receive information;
a central cloud server system comprising a hospital database pool configured to store policyholder information pertaining to customers and pets, a system API, and a web portal interface used to interact with databases in the central cloud server system;
a backend server system comprising a machine-learning API and a machine-learning model configured to store one or more pieces of information concerning veterinary treatments or procedures classified as approved, a business rule engine used to validate and process claim or precertification information upon receipt in real time based upon the approved veterinary treatments or procedures; and
a pet hospital server system comprising one or more computers with a display connected to a Practice Information Management System (PIMS) database configured to store pet hospital customer records and pet medical and billing records.

10. The system of claim 9, further including:
the backend server system configured to transmit data to a database in the central cloud server system to update the policyholder information including a pet name, contact information, a type of pet, and a policy version; and
a scheduler configured to update the policyholder information in the backend server system based on a scheduled update, wherein the update initiates a transfer of data from a main information database to a duplicate database.

11. The system of claim 9, wherein the machine-learning API is configured to mark each item in an invoice as a type of charge, wherein the marking is based upon pre-determined input conditions and machine learned data of prior claims and precertification.

12. The system of claim 11, wherein the backend server system is further configured to initiate a payment to a hospital based upon a calculated covered amount and updating payment information.

13. The system of claim 12, wherein the backend system server transmits the calculated covered amount and a notice of procedure approval to hospital to the one or more computers with a display on the web portal.

14. The system of claim 12, wherein the backend system server transmits the calculated total covered amount and a notice of payment to hospital to the one or more computers with a display.

15. The system of claim 9, wherein the processed claim or precertification information includes:
customer information, including name;
pet information, including type of pet, age, breed, visit diagnosis;
at least one of a date of service and a future date of service;
at least one of a service provided on the date of service and a service estimate provided for the future date of service; and
cost information for the at least one service provided.

16. The system of claim 9, wherein the backend server system further comprises an Extract, Transform, Lead Engine (ETL), utilizes uses ETL packages to transfer data from a main information database to a duplicate database located in the central could server system.

17. The system of claim 9, wherein the pet hospital server system further comprises a hospital service connected to the PIMS database, the hospital service configured to extract data from the PIMS database and transmit data to a web portal.

18. The system of claim 17, wherein the pet hospital server system further comprises an update service connected to the hospital service, update service configured to perform system revisions and updates.

19. The system of claim 9, wherein the one or more computers of the central cloud server system includes a web portal.

20. A computer readable medium with computer executable instructions performing the following steps:
receiving information including a user name, a hospital location, customer information, and request information related to one of the group consisting of a pet insurance claim and a pet insurance precertification;
sending the user sign on credentials including the user name, the hospital location, the customer information, and the request information over a communication network from a veterinary hospital, wherein the user sign on credentials are sent to a central cloud server to retrieve a matching hospital database pool;
retrieving from the matching hospital database pool one of an invoice matching the pet insurance claim and an estimate matching the pet insurance precertification, wherein the one of the invoice and the estimate includes a plurality of item types;
transmitting the request information from the central cloud server to a machine-learning API in a backend system server;
electronically processing the one of the invoice and the estimate in the machine-learning API by marking each item type in the one of the invoice and the estimate, wherein the marking is based upon pre-determined input conditions and data from prior claims and precertifications to create a machine-learning model;
electronically processing the one of the pet insurance claim and the pet insurance precertification in a business rules engine;
calculating a total amount, wherein the total amount is an agreed upon amounts for each item approved by the business rules engine during the step of electronically processing;
storing the agreed upon amount in a backend system server database; and
initiating a payment to the hospital location based upon the calculated total amount.
